(19) United States
(12) Patent Application Publication
(10) Pub. No.: US 2003/0187389 A1
(43) Pub. Date: Oct. 2, 2003

(54) Center support for steerable electrophysiology catheter

(75) Inventors: Steve Morency, Sunnyvale, CA (US); Jeff Jones, Hillsborough, CA (US); Sougata Banerjee, San Jose, CA (US)

Correspondence Address: BINGHAM, MCCUTCHEN LLP, THREE EMBARCADERO, SUITE 1800, SAN FRANCISCO, CA, 94111-4067, US

(73) Assignee: SciMed Life Systems, Inc.

(21) Appl. No.: 10/109,817

(22) Filed: Mar. 29, 2002

(51) Int. Cl. A61M 31/00

(52) U.S. Cl. 604/95.04; 604/524

(57) ABSTRACT

A steerable catheter including a handle assembly, a guide tube assembly, an electrode tip assembly, an electrical cable for providing power to an electrode tip assembly, and plugs, which connect with appropriate conventional catheter control equipment (not shown). The handle assembly includes a steering mechanism adapted to pull steering wires to bend the electrode tip assembly. The electrode tip assembly includes a plastic or composite injection molded center support having a body of varying thickness.

### FIELD OF THE INVENTION

The present invention is related to steerable catheters that can be steered and manipulated within interior regions of the body from a location outside the body and, more particularly, to a center support for the bendable distal tips of such catheters.

### BACKGROUND OF THE INVENTION

Physicians make widespread use of catheters in medical procedures to gain access into interior regions of the body. It is important that the physician can control carefully and precisely the movement of the catheter within the body, especially during procedures that ablate tissue within the heart. These procedures, called electrophysiological therapy, are used for treating cardiac rhythm disturbances.

During such procedures, a physician steers a catheter through a main vein or artery (which is typically the femoral artery or vein) into the interior region of the heart that is to be treated. The physician then further manipulates a steering mechanism to place the electrode carried on the tip of the catheter into direct contact with the tissue that is to be ablated. The physician directs radio frequency energy into the electrode tip to ablate the tissue and form a lesion.

Cardiac ablation especially requires the ability to precisely bend and shape the tip end of the catheter to position the ablation electrode. The tip end of the catheter typically includes a bendable center support comprising a flat piece of sheet metal that acts like a spring. The center support enables the tip end to obtain a desired curve when pulled by a steering wire during use of the catheter and then springs back to a neutral or straight position once tension on the steering wire is released. Because the thickness of the center support tends to be about 0.005 inches thick, catheters using such center supports may be prone to bending or steering problems.

Thus, it is desirable to provide a steerable catheter with a more durable center support without diminishing the steering capabilities of the catheter.

### SUMMARY OF THE INVENTION

The present invention is directed to catheters, usable in both diagnostic and therapeutic applications, that enable a physician to swiftly and accurately steer a distal end of such catheters into a selected curved shape within the body of a patient.

Preferably, a steerable catheter in accordance with the present invention includes a handle, an elongated catheter body, a steering tube preferably formed by an elongate coiled spring and a steering member. The steering tube has a distal end that terminates short of the distal end of the catheter with the steering member extending therefrom. The steering member comprises a center support formed of a injection molded plastic or composite material. The molded center support, having a continuous taper toward its distal end, is bendable in response to external forces to steer the catheter tip and, because of the eleastic recoil characteristics of the composite material, springs back to a neutral or straight position once the external steering forces are released. The distal end of the steerable catheter is preferably provided with an operative component such as a tip electrode. At least one steering wire (and preferably two) is attached to the steering member for transmitting bending force thereto from a steering mechanism.

Further, objects and advantages of the invention will become apparent from the following detailed description and accompanying drawings.

### BRIEF DESCRIPTION OF DRAWINGS

FIG. 1 a perspective view of a catheter and catheter handle assembly in accordance with the invention;

FIG. 2 is a fragmentary exploded disassembled view perspective of the electrode tip assembly of a conventional catheter;

FIG. 3 is a perspective view of a center support of the present invention;

FIG. 4 is a side view of the center support shown in FIG. 4;

FIG. 5 is a perspective view of an alternative embodiment of the center support of the present invention.

### DETAILED DESCRIPTION OF PREFERRED EMBODIMENTS

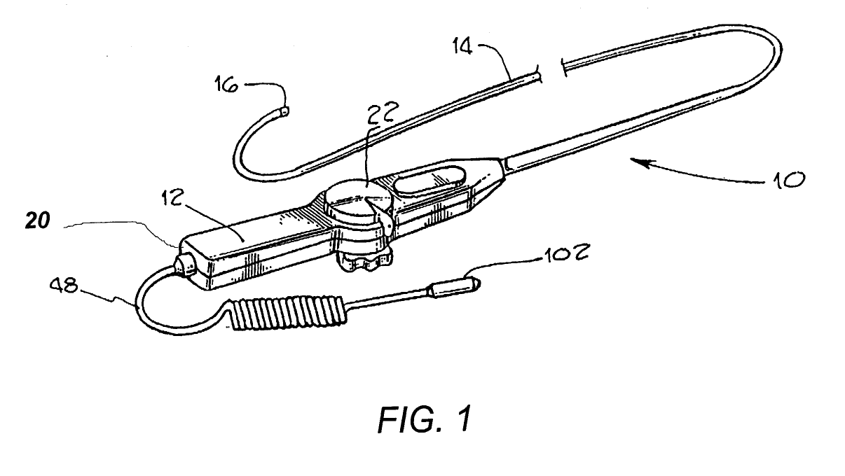

FIG. 1 shows a steerable catheter 10 in accordance with the present invention. As there shown, the catheter 10 includes three main parts or assemblies: the handle assembly 12, the guide tube assembly 14, and the electrode tip assembly 16. The handle assembly 12 includes a steering mechanism 22 which may be, for example, in the form of a rotating cam wheel of the type shown in U.S. Pat. No. 5,195,968 or U.S. Pat. No. 5,891,099, the disclosures of which are incorporated herein by reference. An electrical cable 48 for providing power to an electrode at the distal tip of the catheter attaches to the back end of the handle housing 20. The cable 48 ends with plugs 102 that connect with appropriate conventional catheter control equipment (not shown).

The catheter 10 can be used in many different environments and procedures, such as electrophysiologic therapy in the interior regions of the heart. When used for this purpose, a physician grips the handle assembly 12 to steer the guide tube assembly 14 through a main vein or artery (which is typically the femoral arterial) into the interior region of the heart that is to be treated. The physician then further manipulates the steering mechanism 22 on the handle assembly 12 to place the electrode tip assembly 16 in contact with the tissue that is to be ablated. The physician directs radio frequency energy into the electrode tip assembly 16 to ablate the tissue contacting the electrode tip assembly 16.

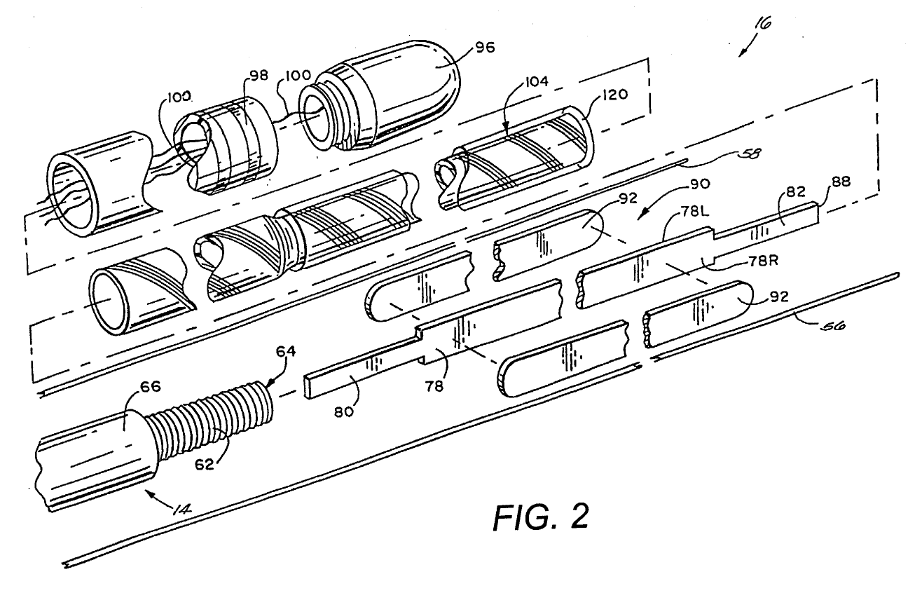

FIG. 2 shows a fragmentary exploded view of the guide tube and distal tip assemblies 14 and 16. As there shown, the guide tube assembly 14 includes a flexible shaft 62 attached to the handle assembly 12. The flexible shaft 62, which comprises a length of stainless steel coiled into a flexible spring, encloses an interior bore 64. A sheath 66 of extruded plastic material containing wire braids encloses the coil 62. The sheath 66 is preferably made from a thermoplastic material, such as a polyurethane, a polyolefin or polyetherpolyamide block copolymer.

Alternatively the shaft 62 may comprises a slotted, stainless steel tube enclosing the interior bore. Further details of such slotted shafts are disclosed in U.S. Pat. No. 5,315,996, the disclosure of which is incorporated by reference.

The electrode tip assembly 16, as shown in FIG. 2, includes a conventional bendable center support 78 and stiffening spring assembly 90. The center support 78 is typically made of stainless steel flat wire stock in an elongated shape about 0.035 inch wide, about 0.005 inch thick and about 3 inches in total length.

The opposite ends of the center support wire 78 are typically cut away to form stepped shoulders 80 and 82. The shoulders 80 and 82 are about 0.024 inch wide and aligned along the centerline of the center support wire 78. Each shoulder 80 and 82 is about 0.12 inch in length. The stepped shoulder 80 on the proximal end of the center support wire 78 fits within the distal end of the flexible guide tube shaft 62 to append the electrode tip assembly 16 to the guide tube assembly 14.

The stiffening spring assembly 90 stiffens the center support wire 78 by sandwiching the center support wire 78 between two leaf springs 92. Each leaf spring 92 is made of stainless steel flat wire stock in an elongated shape that is about 0.035 inch wide and about 0.0025 inch thick. The leaf springs 92 typically extend from just after the distal shoulder 80 to about the midplane of the center support wire 78. The thickness and/or stiffness of the leaf springs 92 are typically manipulated to provide asymmetrical right and left bending profiles for the distal tip assembly 16.

Steering wires 56 and 58 extend from the steering mechanism 22 in the handle 12 through the interior bore 64 of the flexible shaft 62 to connect to the bendable center support 78. A distal end of the left steering wire 58 is soldered to the left face 78L of the center support wire 78, while a distal end of the right steering wire 56 is soldered to the right face 78R of the center support wire 78. When pulled by the steering mechanism 22, the left steering wire 58 bends the center support wire 78 to the left and the right steering wire bends the center support wire 78 to the right.

In the illustrated embodiment, the distal end of the electrode tip assembly 16 carries an ablation tip electrode 96 and three ring electrodes 98. Interior conducting wires 100 are connected to the tip electrode 96 and the three ring electrodes 98. The conducting wires 100 extend along the center support wire 78, through the interior bore of the guide tube shaft 62, and into the handle housing 20 to join the cable 48 that extends from the rear of the housing 20. The conducting wires 100 transfer electrical current from the ring electrodes 98 indicative of electrical activity within the heart. The conducting wires 100 also transfer radio frequency energy to the tip electrode 96 to carry out ablation procedures within the heart.

A reinforcing sleeve assembly 104, which may be formed out of Kevlar, medical grade TFE, or the like, holds the steering wires 56 and 58 in close intimate contact against the main support wire 78. Isolation of the conducting wires 100 from the steering wires 56 and 58 prevents kinking and chafing of the conducting wires 100 during bending operations.

An outer tube 120 covers the reinforcing sleeve assembly 104. The tip electrode 96 is soldered to the center support 78 and ring electrodes 98 are attached to the conducting wires 100 and joined to the outer tube 120 by conventional methods to complete the electrode tip assembly 16.

As noted above in the background, the conventional bendable center support is prone to collapse and, thus, failure due to the typical thickness associated with the center support wire 78. In addition, the conventional center support requires multiple parts. i.e., the center wire plus leaf springs, to achieve the desired bending profile. A bendable center support in accordance with the present invention, however, is preferably formed as a single component from injected molded plastic or composite materials that is capable of performing functions previously requiring multiple components. The strength of today's composites and plastics provide many advantages over conventional stainless steel components. The ability to vary the thickness as well as create custom shapes to suit the needs of the various distal end 16 configurations and bending profiles tends to provide much more reliable catheter performance at lower costs. Laminates or carriers suitable for use in the present invention could be made of any of the following materials: Udel P-1700, Viatrx, PES 200P, Ultem, Torlon, Ryton Peek, Avimid, CM-X, and the like. The reinforcing fibers could be made of: fiberglass, graphite, aramid, polyethylene, boron, silicon carbide, silicon nitride, silica, alumina, alumina silica, and the like. Use of materials such as polyurethane or composites that include silicon rubber or latex is preferable to insure that the center support exhibits an appropriate elastic recoil characteristic necessary to enable the center support to spring back to a neutral or straight position after bending.

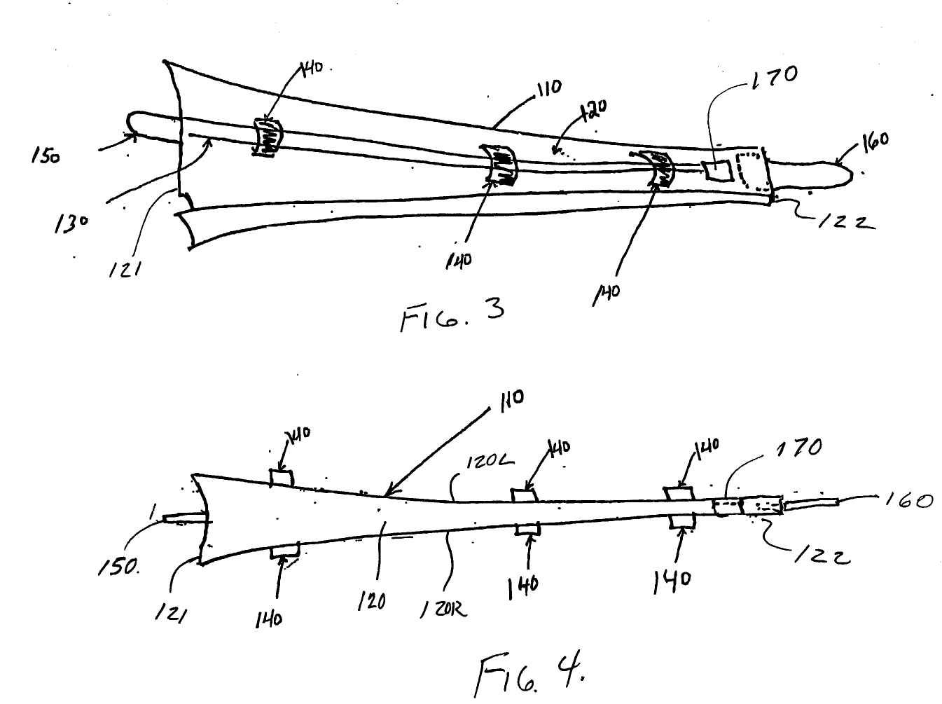

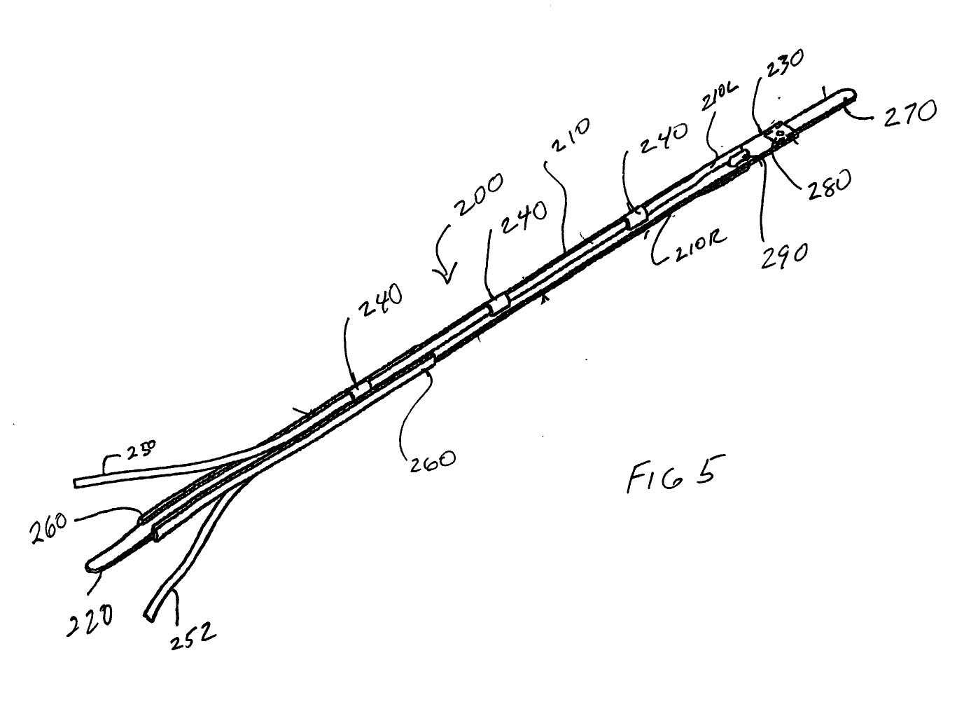

As shown in FIGS. 3 and 4, a preferred embodiment of the molded center support 110 of the present invention includes a body 120 formed from injection molded plastic or composite material. The body 120 preferably comprises a continuous taper from a proximal end 121 to a distal end 122 of the body 120. The taper may be formed along the width or thickness, or as shown in the illustrated embodiment, along both the width and thickness of the body 120. As such, the stiffness of the center support 110 tends to be greatest at its proximal end 121 where the thickness of the body 120 is greatest.

The molded center support 110 includes proximal and distal tongue shaped tangs 150 and 160 extending from the proximal and distal ends 121 and 122 of the body 120. Preferably, the distal tang 160, to which the ablation electrode 96 is attached, comprises a stainless steel tang insert molded into the body 120. The proximal tang 150 may be integrally formed with the body or may comprise a stainless steel tang insert molded into the body 120. The proximal tang 150 is inserted into interior bore 64 of the shaft 62 and, thus, used to append the distal tip assembly 16 to the guide shaft assembly 14.

Wire channels 130 are formed in the right and left faces 120R and 120L of the body 120. A series of wire retaining loops 140 may be integrally formed with or attached to the body 120 at graduated locations along the channels 130 in the right and left faces 120R and 120L of the body 120. The loops 140 are used to retain the steering wires 56 and 58 in the wire channels 130. A window 170 may be formed through the body 120 at a point just beyond the last loop 140 toward the distal end 122 of the body 120 and used to loop a single steering wire there through. Alternatively, the steering wires 56 and 58 may be mechanically attached, i.e., soldered, spot welded, crimped or the like, to an attachment component (not shown) insert molded into the body.

The thickness of the molded center support 110 may be varied to advantageously compensate and eliminate the need for the leaf springs 92 used with the conventional center support wires 78 to support various bending profiles. Furthermore, the wire retaining loops 140 advantageously capture the steering wires 56 and 58 and retain the steering wires 56 and 58 in place within the wire channels 130. As such, the retaining loops 140 enable the elimination of the Kevlar reinforcing sleeve 104 used with the conventional bendable support 78 to keep the whole steering assembly in place. Furthermore, because features capable of holding a continuous wire in place may be integrally or insert molded into the body 120 of the center support 110, the need to mechanically attach the steering wires 56 and 58 to the center support may advantageously be eliminated.

In operation, pulling on the left steering wire 58 will cause the center support 110 to bend to the left, while pulling on the right steering wire 56 will cause the center support 110 to bend to the right.

Turning to FIG. 5, an alternate embodiment of the molded center support 200 is there shown to include an elongate body 210 with proximal and distal ends 220 and 230. The body 210 is preferably injection molded from plastic or a composite material. Preferably, the plastic or composite material has sufficient elastic coil characteristics to enable the center support to spring back to a neutral position after bending. To accommodate desired curve or bending profiles in the distal tip 16 and, thus, compensate for and eliminate the need for leaf springs, the thickness of the body 210 may be varied over the entire length of the body 210, thus varying the stiffness of the body 210 over its entire length.

The molded center support 200 preferably comprises a series of wire guides 240 at graduated locations along the right and left faces 210R and 210L of the body 210. The guides 240 may be molded integrally or insert molded with the body 210. The guides 240 act to guide and retain steering wires 250 and 252 along the right and left faces 210R and 210L of the body 210. Preferably, the body 210 includes a pair of opposing side rails 260 to guide the steering wires to the first guide 240 along each side of the body 210. At a point after the last guide loop 240, on both faces toward the distal end 230 of the body 210, is a connector 290 to which the steering wires 250 may be crimped, pinned, soldered, or the like. The connector 290 may be insert molded with the body 210.

A stainless steel tang 270, preferably tongue-shaped, may be insert molded into the distal end 230 of the body. Alternatively, the distal tang 270 may be attached by spot welding or the like, to an anchor 280 insert molded adjacent the distal end 230 of the body 210. The proximal end 220 may be formed in appropriate shape and size to be inserted into the interior bore 64 of the shaft 62. Alternatively, a proximal tang may be integrally or insert molder with the body 210.

In operation, pulling on the left steering wire 250 will cause the center support 200 to bend to the left, while pulling on the right steering wire 252 will cause the center support 200 to bend to the right.

While various preferred embodiments of the invention have been shown for purposes of illustration, it will be understood that those skilled in the art may make modifications thereof without departing from the true scope of the invention as set forth in the appended claims including equivalents thereof.

What is claimed:

1. A center support for a catheter having an elongated tubular body bendable adjacent to its distal end in response to an external force, and a proximal end attached to a handle for manipulating the tubular body by applying the external force thereto, comprising,
an injection molded body having distal and proximal ends wherein the body includes a continuous taper in a first dimension along the length of the body from the proximal end to the distal end, and
first and second tangs extending from the proximal and distal ends of the body.

2. The center support of claim 1 wherein the body is formed from injection molded plastic.

3. The center support of claim 1 wherein the body is formed from injection molded composite material.

4. The center support of claim 1 wherein the body includes a continuous taper in a second dimension along the length of the body from the proximal end to the distal end.

5. The center support of claim 4 wherein the proximal end has a greater thickness and width than the distal end.

6. The center support of claim 1 further including first and second wire channels formed in opposing faces of the body and adapted to receive a steering wire.

7. The center support of claim 6 further including a plurality of wire retaining loops positioned in graduated spacings along the first and second wire channels.

8. The center support of claim 1 wherein the distal tang is formed from stainless steel.

9. The center support of claim 8 wherein the stainless steel distal tang is insert molded into the body.

10. The center support of claim 1 wherein the body includes a window formed there through adjacent the distal end of the body to enable passage of a continuous steering wire.

11. A center support for a catheter having a bendable distal end, comprising,
an injection molded elongate body of varying thickness having distal and proximal ends and first and second faces,
first and second tangs extending from the proximal and distal ends of the body, and
a plurality of guides spaced along first and second faces, the plurality of guides being adapted to receive and retain first and second steering wires adjacent the first and second faces.

12. The center support of claim 11 wherein the body is formed from injection molded plastic.

13. The center support of claim 11 wherein the body is formed from injection molded composite material.

14. The center support of claim 1 further including first and second sets of wire channels formed on first and second faces of the body and adapted to guide a steering wire toward a first guide of the plurality of guides on the first and second faces of the body.

15. The center support of claim 14 further including a steering wire connector positioned toward the distal end of the body.

16. The center support of claim 11 wherein the distal tang is formed from stainless steel.

17. The center support of claim 16 wherein the stainless steel distal tang is insert molded into the body.

18. A steerable catheter comprising
a handle,
an elongate tubular body extending from the handle, the tubular body having distal and proximal ends and being bendable adjacent its distal end,
an injection molded center support positioned in the tubular body adjacent its distal end, the center support having first and second faces and a plurality of wire retaining loops spaced along the first and second faces,
a steering mechanism in the handle, and
first and second steering wires extending from the steering mechanism through the tubular body and the plurality of retaining loops on the first and second faces of the center support.

19. The catheter of claim 18 wherein the center support is formed from plastic or a composite material.

20. The catheter of claim 19 wherein the first and second steering wires form a single continuous steering wire.

21. The catheter of claim 20 wherein the center support includes a window formed adjacent the distal end of center support and through which the continuous steering wire passes.

22. The catheter of claim 19 wherein the first and second steering wires include distal ends attached to the distal end of the center support.

23. The catheter of claim 22 wherein the center support includes a wire connector positioned adjacent the distal end of the support.

24. The catheter of claim 18 wherein the center support includes a continuous taper in a first dimension along the length of the center support from its proximal end to its distal end.

25. The catheter of claim 18 wherein the center support includes first and second tangs extending from the proximal and distal ends of the body.

26. The catheter of claim 24 wherein the center support includes a continuous taper in a second dimension along the length of the center support from its proximal end to its distal end.

27. The catheter of claim 26 wherein the proximal end has a greater thickness and width than the distal end.

28. The catheter of claim 18 further including first and second wire channels formed in first and second opposing faces of the center support and adapted to receive a steering wire.

29. The catheter of claim 25 wherein the second tang extending from the distal end of the center support is formed from stainless steel.

30. The catheter of claim 29 wherein the stainless steel distal tang is insert molded into the center support.

31. A center support for a steerable catheter comprising an injection molded body having distal and proximal ends wherein the body includes a continuous taper in a first dimension along the length of the body from the proximal end to the distal end.

32. The center support of claim 31 wherein the body is formed from injection molded plastic.

33. The center support of claim 31 wherein the body is formed from injection molded composite material.

34. The center support of claim 31 wherein the body includes a continuous taper in a second dimension along the length of the body from the proximal end to the distal end.

35. The center support of claim 34 wherein the proximal end has a greater thickness and width than the distal end.

36. The center support of claim 35 further including first and second wire channels formed in opposing faces of the body and adapted to receive a steering wire.

37. The center support of claim 36 further including a plurality of wire retaining loops positioned in graduated spacings along the first and second wire channels.

38. The center support of claim 37 further comprising first and second tangs extending from the proximal and distal ends of the body, wherein the distal tang is formed from stainless steel.

39. The center support of claim 38 wherein the stainless steel distal tang is insert molded into the body.

40. The center support of claim 31 wherein the body includes a window formed there through adjacent the distal end of the body to enable passage of a continuous steering wire.
